(19) United States
(12) Patent Application Publication
(10) Pub. No.: US 2003/0225378 A1
(43) Pub. Date: Dec. 4, 2003

(54) Mixing device for surgical sealants, and method thereof

(75) Inventors: James Wilkie, Melrose, MA (US); James Rolke, Fitzwilliam, NH (US); Luis Burzio, Andover, MA (US); Gary Ayotte, Newburyport, MA (US)

Correspondence Address: TESTA, HURWITZ & THIBEAULT, LLP, HIGH STREET TOWER, 125 HIGH STREET, BOSTON, MA, 02110, US

(73) Assignee: Surgical Sealants, Inc., Boston, MA (US)

(21) Appl. No.: 10/423,768

(22) Filed: Apr. 25, 2003

Related U.S. Application Data
(60) Provisional application No. 60/375,683, filed on Apr. 26, 2002.

(51) Int. Cl. A61M 5/315

(52) U.S. Cl. 604/221

(57) ABSTRACT

What is described herein is a mixing device having a plurality of chambers and at least one transferor for mixing components of a tissue sealant.

### RELATED APPLICATIONS

This application claims benefit of U.S. provisional application serial No. 60/375,683 filed on Apr. 26, 2002.

### BACKGROUND OF THE INVENTION

Fluid and/or gaseous leaks can result from surgeries involving vascular, pulmonary, thoracic, spinal, meningeal, neural, hepatic, lymphatic, digestive, oncological, gynecological and renal tissues. The current standard of care involves the use of hemostats such as thrombin, gelatin and fibrin glue for diffuse bleeding, or the placement of drains until wound resolution.

Tissue sealants are topically applied fluid or gel media used to repair defects such as perforations or tears in tissues such as, for example, the lungs, vessels, the dura mater, and intestines. Tissue sealants are generally made ready for immediate use in surgery by first mixing together two or more components of the tissue sealant just prior to their application to tissue, followed by rapid application of the sealant to the tissue before the sealant cures and is no longer suitable for application to a tissue surface.

Typically, the preparation of the sealant occurs during the surgical procedure. Usually, one component of a sealant is provided in a powder form and must be dissolved in a diluent prior to mixing with the other sealant components. The powder is typically packaged in a vial. The seal of the vial must be broken by the user with a needle or other device and the diluent is injected into the vial to dissolve the powder. This process can make the sealant difficult to use, is fraught with delays, and is prone to error. Moreover, once mixed, the sealant begins to rapidly cure and if not used immediately, the sealant cures and is no longer useful as a sealant. There is, therefore, a need for devices and methods for preparing tissue sealants that are easy to mix, do not elicit severe adverse host reactions, and have a predictable cure rate.

### SUMMARY OF THE INVENTION

The invention provides devices and methods for mixing elements of a composition. According to the invention, a device comprises three separate housings, which may be chambers. A device of the invention further comprises at least one mixing element for passing elements of a composition reciprocally between the housings. In use, a first element of a composition is placed in a first housing, a second element of the composition is placed in a second housing, and the two elements are mixed by reciprocal transfer through a third housing. The third housing is typically placed between the first and second housings to allow reciprocal passage of the elements between the housings for mixing. The result is a composition that is a mixture of the elements. In certain embodiments, the mixture contains more than two components. In that case, multiple components are placed into the housings. However, it is preferred that reactive components are sequestered until mixing is desired.

A device of the invention is useful for mixing components of a composition, such as a tissue sealant for sealing fluid or gas leaks from tissues, for example, the lungs, vessels, intestines or neural tissues. In one embodiment, a device of the invention comprises a first chamber comprising a first lumen for holding a first component. The device further comprises a second chamber comprising a second lumen for holding a second component. The first and second lumens may be in fluid communication. In a preferred embodiment a device of the invention comprises a third chamber comprising a third lumen, which may be in fluid communication with the other two lumens. Another feature of a device of the invention is a transferor or mixing element for reciprocal transfer of at least one of the first and second components between at least two of the first, second, and third chambers.

A third housing of a device of the invention may hold a third component. In one embodiment, a device of the invention further comprises a removable barrier interposed between at least two of the housings. The removable barrier may comprise a valve interposed between at least two of the housings. In one embodiment, the valve is a stop-cock.

In another embodiment, a soluble plug or an insoluble breakable membrane is used to divide one or more of the first, second, and third housings. Optionally, the first housing is in fluid communication with the second housing. In a further feature, the second housing is in fluid communication with the third housing. Optionally, the first housing is in fluid communication with the third housing.

A mixing element of a device according to the invention is selected from a plunger, a piston, a bladder, a pump, a propeller, an intraluminal ball or any combination thereof. In a particular embodiment according to the invention, the transferor or mixing element is a first plunger joined to the first housing and a second plunger joined to the third housing for reciprocally transferring at least one of the first and second components between the first housing and the third housing through the second housing. In another feature, at least two of the housings are detachable.

A preferred device of the invention comprises a slideably mounted member. The slideably mounted member contains a bore, the bore forming the second housing. Optionally, the second housing is further divided into a plurality of housings.

In one embodiment according to the invention, at least two of the housings are syringes. In another embodiment, the removable barrier is a soluble plug. In yet another embodiment, the removable barrier is an insoluble breakable membrane. In yet another embodiment, one or more housings include an obstruction to create turbulent flow to enhance mixing efficiency.

Another aspect of the invention relates to a system for preparing a tissue sealant with a predetermined cure rate. In one embodiment according to the invention, a system comprises a first chamber including a lumen, and a first mixture held in the first chamber. The first mixture, in one embodiment, includes an adhesive matrix and a cross-linker. The system also includes a second chamber. According to one embodiment, the second chamber is detachably fitted to the first chamber. In one embodiment, a second component is held in the second chamber for adjusting the pH of the first mixture held in the first chamber. In another embodiment according to the invention, the first mixture from the first chamber is mixed with the second component from the second chamber to form a second mixture and the second component adjusts the pH of the second mixture to form a sealant having the predetermined cure rate.

In one embodiment according to the invention, the second component held in the second chamber is a buffer. The buffer is selected from the group consisting of an ion exchange resin, organic acids, inorganic acids, acid exchange resins, organic bases, inorganic bases and alkaline exchange resins. Optionally, the buffer is selected from citric acid, phosphate, succinate, acidic salts, and morpholinoethanesulfonic acid. As another example, in another embodiment according to the invention, the pH of the first mixture is in the range of about 7-13. In this embodiment, the second component of the system lowers the pH of the second mixture to control the cure rate of the second mixture. In yet another embodiment, the pH of the first mixture is in the range of about 1-7. In this embodiment, the second component raises the pH of the second mixture to control the cure rate of the second mixture.

In another embodiment, the first mixture includes, for example, a carbodiimide crosslinker. In another embodiment, the lumen of the second chamber is coated with the second component.

Another aspect of the invention relates to a method for preparing a tissue sealant with a predetermined cure rate. In one embodiment according to the invention, the method includes fitting a first chamber holding a first mixture to a second chamber. In one embodiment, the mixture in the first chamber includes an adhesive matrix and a cross linker at about pH 7-13. In one embodiment, the method includes introducing the first mixture from the first chamber into the second chamber holding a buffer. The buffer in the second chamber adjusts the pH of the first mixture to a predetermined pH level. The method further includes the step of mixing the first mixture and the buffer in the second chamber to form a sealant having a predetermined cure rate.

These and other objects, along with advantages and features of the present invention herein disclosed, will become apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE FIGURES

The foregoing and other objects, features and advantages of the present invention disclosed herein, as well as the invention itself, will be more fully understood from the following description of preferred embodiments and claims when read together with the accompanying drawings. The drawings are not necessarily to scale, emphasis instead generally being placed upon illustrating the principles of the invention.

FIG. 1 is a schematic side view of a mixing device according to an illustrative embodiment of the invention.

FIGS. 2-4 are schematic side views of other embodiments of a mixing device according to an illustrative embodiment of the invention.

FIG. 5 is a schematic side view of a three chamber mixing device including a side port according to an illustrative embodiment of the invention.

FIG. 6 is a schematic side view of a three chamber mixing device according to an illustrative embodiment of the invention.

FIGS. 7A-7C depict a method for mixing the components of a sealant in a mixing device according to an illustrative embodiment of the invention.

FIG. 8 is a schematic side view of a device including a fourth chamber formed by a barrier according to an illustrative embodiment of the invention.

FIGS. 9A and 9B are schematic side views of a mixing device including a rotationally mounted bore according to an illustrative embodiment of the invention.

FIG. 10A is a schematic side view of a mixing device including a slideable member according to an illustrative embodiment of the invention.

FIG. 10B is a schematic side section view of a mixing device including a slideable member in the closed position according to an illustrative embodiment of the invention.

FIG. 10C is a schematic side view of a mixing device including a slideable member according to an illustrative embodiment of the invention.

FIG. 10D is a schematic top view of one embodiment of a casing member illustrated in FIGS. 10A, 10B, and 10C.

FIG. 10E is a schematic cross section of the casing member illustrated in FIG. 10D taken along line 10E-10E of FIG. 10D.

FIG. 10F is a schematic end view of one embodiment of the casing member illustrated in FIG. 10D.

FIG. 10G is a schematic end view of one embodiment of the casing member illustrated in FIG. 10D.

FIG. 10H is a schematic cross section of the casing member illustrated in FIG. 10D taken along line 10H-10H of FIG. 10D.

FIG. 10I is an isometric schematic depiction of one embodiment of a luer lock fitting that can be used in association with the casing member illustrated in FIG. 10D.

FIG. 10J is a schematic side view of one embodiment of the luer lock fitting illustrated in FIG. 10I.

FIG. 10K is a schematic cross section of the luer lock fitting illustrated in FIG. 10J taken along line 10K-10K of FIG. 10J.

FIG. 10L is a schematic front view of a slideable member according to an illustrative embodiment of the invention.

FIG. 10M is a schematic side section view of the slideable member illustrated in FIG. 10L taken along line 10M-10M of FIG. 10L.

FIG. 10N is a schematic side view of the slideable member illustrated in FIG. 10L.

FIG. 10O is a schematic top view of the slideable member illustrated in FIG. 10L.

FIG. 10P is a schematic side section view of a mixing device including a slideable member in an open position according to an illustrative embodiment of the invention.

FIGS. 11A and 11B are schematic side views of a mixing device including a plurality of chambers separated by a stationary tab according to an illustrative embodiment of the invention.

FIG. 12 is a schematic side view of a mixing device including a mixing chamber according to an illustrative embodiment of the invention.

### DETAILED DESCRIPTION OF THE INVENTION

In general, the invention provides a mixing device for mixing components of a composition, for example, a tissue sealant, and methods of use. In one illustrative embodiment, the sealant includes a mixture of albumin, a lipid, water, an enzyme, a surfactant and a cross-linker, for example, as described in U.S. application Ser. No. 09/747,293, the entirety of which is incorporated by reference herein. All of the embodiments of the device and method of the invention described herein have in common a series of chambers and mixing elements, e.g., transferors that allow for the controlled, rapid mixing of components of the tissue sealant.

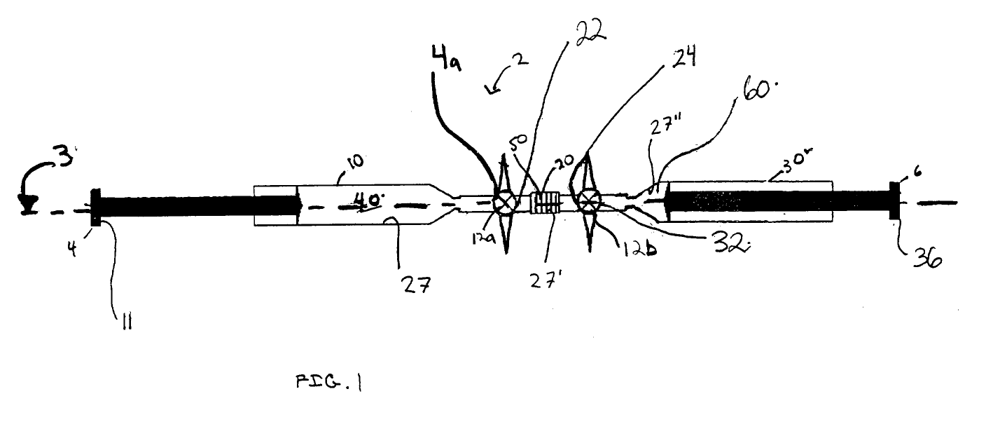

Referring now to FIG. 1, in the illustrative embodiment, the device 2 according to the invention is a closed system including a first housing 10, a second housing 20, and a third housing 30 all in series. Each housing can be a chamber, with each chamber comprising a lumen 27. In the illustrative embodiment, the lumen 27 of chamber 10 is in direct fluid communication with the lumen 27′ of the second chamber 20, and the lumen 27′ of the second chamber 20 is in direct fluid communication with the lumen 27″ of the third chamber 30. According to one feature of this embodiment, the contents of the first chamber 10 and the contents of the second chamber 20 are reciprocally transferable between the first chamber 10 and the third chamber 30 through the second chamber 20, i.e., the contents may be moved back and forth between one chamber to another. In the illustrative embodiment, the second chamber 20 is a closed chamber except for end 22 and end 24 of the second chamber 20. The ends 22, 24 are in fluid communication with the first chamber 10 and the third chamber 30, respectively. Embodiments of the chambers include, for example, a tube, a cylinder, a bladder, or a syringe. In one exemplary embodiment according to the invention, the device 2 includes at least one mixing element, i.e., transferor 11 such as a pump or a plunger, for transferring the contents of one chamber to another chamber. According to one feature, the mixing device 2 includes more than one mixing element, for example, a first transferor 11 that is operatively joined to the first chamber 10, and a second transferor 36 that is operatively joined to the third chamber 30. According to another feature of the invention, the device 2 includes at least one removable barrier interposed between at least two of the chambers. The barrier can be a valve 12a positioned, for example, between the first chamber 10 and the second chamber 20, and optionally a second barrier, e.g., a valve 12b positioned, for example, between the second chamber 20 and the third chamber 30. The valves 12a, 12b enable fluid communication between the lumens 27, 27′, 27″ of the chambers to be selective. The barrier can also be a gas, an insoluble breakable membrane, or a soluble plug (later described).

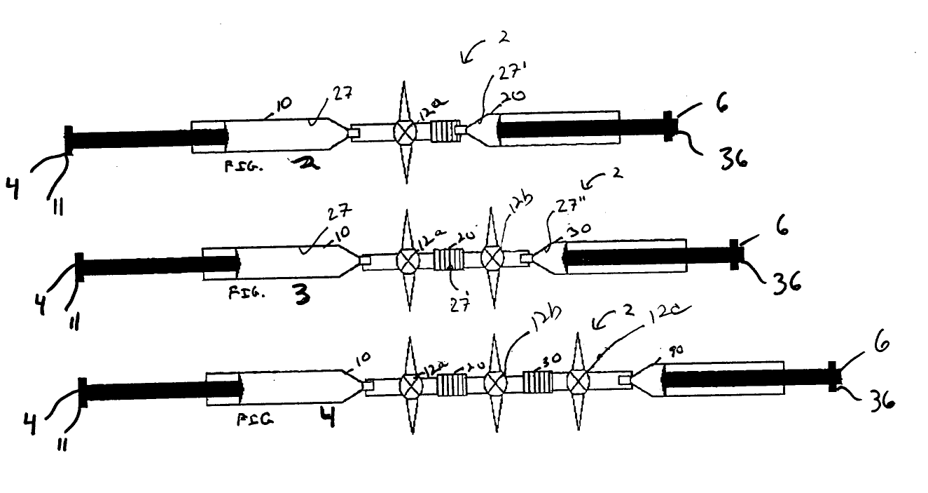

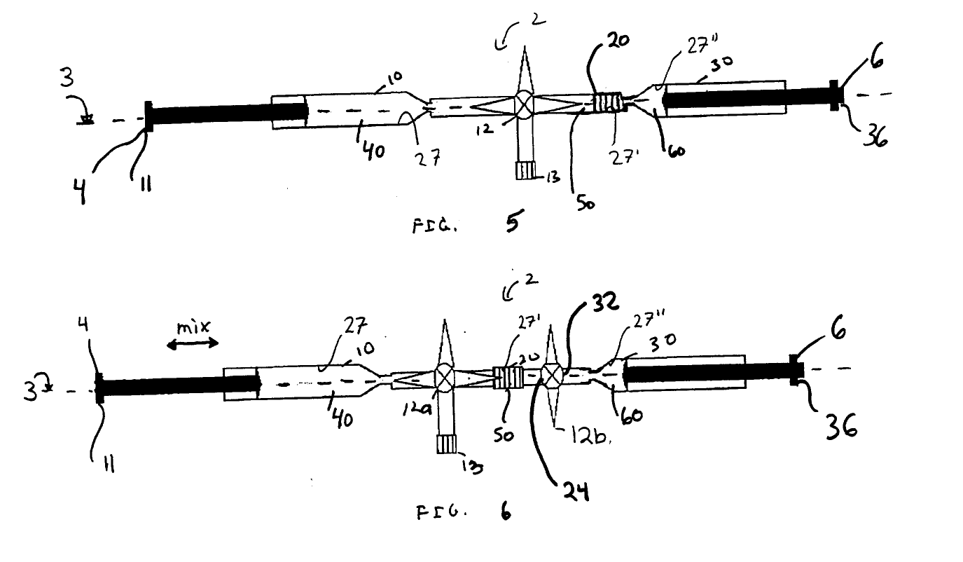

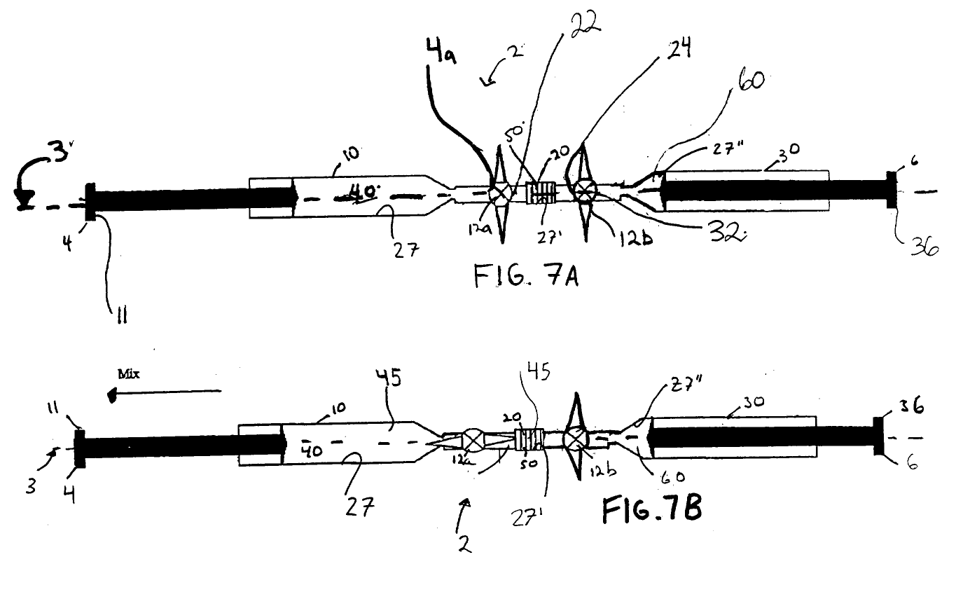

FIGS. 2-4 depict a mixing device 2 including a plurality of housings or chambers according to an illustrative embodiment of the invention. In the illustrative embodiment, the device 2 includes a first chamber 10 and a second chamber 20 (FIG. 2), a first chamber 10, a second chamber 20, and a third chamber 30 (FIG. 3), a first chamber 10, a second chamber 20, a third chamber 30, and a fourth chamber 90 (FIG. 4). In other embodiments, the device 2 includes more than four chambers. According to one embodiment of the invention, any of the chambers may be detached from the device 2. Alternatively, the one or more chambers are fixed to the device 2, and are thus not detachable from the device 2.

The housings, for example, the first chamber 10, second chamber 20, and third chamber 30 may be manufactured from materials such as metal, metal alloys, ceramics, glass, or plastics, such as polyvinylchloride or polyethylene, preferentially, polypropylene or polycarbonate. In a particular embodiment according to the invention, illustrated in FIG. 1, the first chamber 10 and the third chamber 30 are each a hypodermic-like syringe.

According to the illustrative embodiment, a valve 12 is operatively positioned between contiguous housings or chambers. For example, the illustrative embodiment of the device shown in FIG. 4 includes three valves 12. A first valve 12a is positioned between the first chamber 10 and the second chamber 20. A second valve 12b is positioned between the second chamber 20 and the third chamber 30. A third valve 12c is positioned between the third chamber 30 and the fourth chamber 90. Valves positioned between the chambers allow selective fluid communication between the lumens of the chambers.

In one embodiment, the device 2 includes three barrier-free chambers. At least two of the chambers hold a different sealant component. The device further includes a mixing element. The three chambers are assembled to permit fluid communication between the chambers and mixing of the components just prior to use.

Referring again to FIG. 1, according to the illustrative embodiment, the device 2 includes a first end 4 and a second end 6. In a particular embodiment, the second end 6 is opposite to the first end 4 along a longitudinal axis indicated by arrow 3 between the first end 4 and the second end 6. The first housing 10 is positioned at the first end 4 of the device 2. The first valve 12a is positioned between the first housing 10 and the second housing 20 on the end 4a of the first housing 10 that is opposite to the first end 4 of the device 2. In one illustrative embodiment, the first valve 12a is a two way stopcock operatively joined to the first housing 10 and to the first end 22 of the second housing 20. In the same embodiment, the second valve 12b is also a two-way stopcock that is operatively joined to the second end 24 of the second housing 20 and the first end 32 of the third housing 30.

FIG. 5 depicts a three housing device according to the invention including a three-way valve. In the illustrative three chamber embodiment, one or more of the valves 12 are a three-way valve, for example, including a side port 13. With reference to FIG. 6, the device includes a second valve 12b, for example, a stopcock, shown in the closed position. The three-way valve 12a enables the contents of the first housing, i.e. the first chamber 10 and the contents of the second housing, i.e. the second chamber 20 to be reciprocally transferred between the first chamber 10 and the second chamber 20 with the use of the transferor 11 by allowing air to bleed from the otherwise closed system. A three-way valve also adds an additional port 13 for the introduction of another component to modify the sealant or adhesive. In one non-limiting example, as described in greater detail below, if a longer cure time of the sealant is required, the pH could be adjusted by adding a buffer component via the additional port 13 in the three-way valve. Thus, according to the invention the device 2 may have a plurality of valves of a variety of types, e.g., two-way and three-way valves.

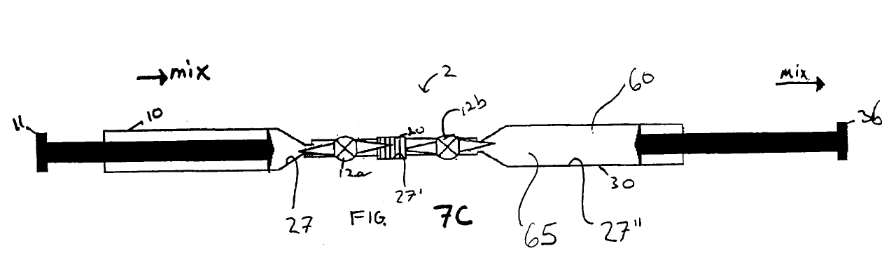

FIGS. 7A-7D depict a method for mixing a tissue sealant according to an illustrative embodiment of the invention. In one aspect, the invention provides a method for mixing the components of a tissue sealant in a mixing device 2. The mixing device 2 includes one or more housings which hold one or more of the tissue sealant components. The housings can be chambers including lumens, where each lumen can hold one or more tissue sealing components. In one embodiment, for example, a first component 40 such as an albumin solution, described in U.S. Ser. No. 09/747,293 and incorporated by reference herein, is held in the first chamber 10 of the mixing device 2. In the illustrative embodiment shown in FIG. 7A, the first chamber 10 is a syringe. The first valve 12a is operatively joined to the first chamber 10 and is in a closed position as illustrated in FIG. 7A. Referring now to FIG. 7B, for example, the first valve 12a is moved from the closed position (FIG. 7A) to an opened position shown in FIG. 7B. When the valve is in the open position, the first component 40 and a second component 50, for example a diluent, such as water, held in the second chamber 20 begin to mix in the lumen 27 of the first chamber 10 and the lumen 27′ of the second chamber 20 forming a first mixture 45. According to the illustrative embodiment, the first mixture 45 is rapidly reciprocally transferred between the second chamber 20 and the first chamber 10 by a mixing element, e.g., a transferor 11. The transferor 11 can be a plunger, a piston, a bladder, an intraluminal ball, a propeller or a pump operatively joined to the first chamber 10. To enable the first mixture 45 to be reciprocally transferred between the second chamber 20 and the first chamber 10, an escape for air, i.e. a vent, must be provided. For example, in one embodiment a three-way valve located between the first chamber 10, and the second chamber 20 (not shown) may provide the vent. Alternatively, if a vent is not provided, the first mixture 45 can be further mixed by aspirating the mixture into the first chamber 10 as illustrated in FIG. 7B.

Referring now to FIG. 7C, the second valve 12b is moved from a closed position (FIG. 7A) to an opened position. The first mixture 45 contained in the first chamber 10 and the second chamber 20 can then be pushed through the second valve 12b and into the third chamber 30 by the transferor 11, for example a plunger 11, or aspirated by the transferor 36, for example, the plunger 36 operatively joined to the third chamber 30. In the lumen 27″ of the third chamber 30, the first mixture 45 is contacted with a third component 60, for example, a cross-linker, such as, 1-ethyl-3-(3dimethylaminopropyl) carbodiimide HCl (EDC) to form a second mixture 65. The second mixture 65 may be reciprocally transferred by the plunger 11 and the plunger 36 between the third chamber 30 and the second chamber 20 or the first chamber 10 to further mix by shearing the second mixture 65, i.e., to mix the first component 40, the second component 50 and the third component 60, to form the tissue sealant. More specifically, referring to FIG. 7C, for example, plunger 11 is depressed manually to transfer the components within the first chamber 10 into the second chamber 20 and third chamber 30. Simultaneously, plunger 36 of the third chamber 30 is pushed out of the chamber 30. The plunger 36 is then depressed to reciprocally transfer the contents of the third chamber 30 between the third chamber 30 and the first chamber 10 through the second chamber 20 while plunger 11 of the first chamber 10 is simultaneously pushed out of chamber 10 (not shown). The number of reciprocal transfers between the first chamber 10, the second chamber 20, and the third chamber 30 depends on the number of transfers needed to thoroughly mix the contents of the chambers. In a particular embodiment, fifteen (15) transfers are needed to mix the components of the chambers to make the tissue sealant. After mixing, the tissue sealant is ready to be applied to a tissue defect such as a perforated lung, vessel, neural tissue or intestine. The disclosed number of chambers, tissue sealant components, and transfers are exemplary and are not intended to be limiting.

In another exemplary embodiment of this aspect of the invention, the device 2 further includes a fourth housing or chamber 90. In one embodiment, the fourth chamber 90, illustrated in FIG. 8, is formed by a barrier 72 such as a plug made from a soluble material. Alternatively the fourth chamber 90 may be formed by a barrier 72 such as an insoluble breakable membrane, for example, that will break upon application of a force to allow sealant components to mix. The barrier 72, such as the soluble plug or insoluble breakable membrane is positioned in a chamber, for example, the third chamber 30 to divide the third chamber 30 into two chambers, i.e., the third chamber 30, and the fourth chamber 90. If a soluble plug is used to form the fourth chamber 90, two powdered components of the sealant may be separated. If an insoluble breakable membrane is used to form the fourth chamber 90, a liquid may be separated from either another liquid or a powder. If a plug is used to divide a chamber, the size of the plug 72 depends on the material used to make the plug 72 and the dimensions of the chamber. In one embodiment of this aspect of the invention, the plug 72 is made from about 0.5 to 6 mg, preferably about 2 mg to 4 mg of a lyophilized protein, e.g., albumin, e.g., bovine serum albumin (BSA). Other materials used to make the plug 72 include, but are not limited to, salts and inert polymers like starch and lipids.

If an insoluble breakable membrane 72 is used to divide a housing or chamber, the insoluble breakable membrane 72 may be made from polymeric materials such as polyethylene, particularly low molecular weight polyethylene, polypropylene, polycarbonate, polybutadiene, polybutadiene-nitrile, polystyrene, polyester, polyamide, polyisoprene, rubber, latex, nitrile, chloroprene, polyvinyl chloride, viton made by DuPont Dow Elastomers L.L.C. (Wilmington, Del.), or foil. The material used to make the barrier 72 should not interfere with the function of the tissue sealant. The number of housings or chambers that may be made by one or more barriers 72 or by the addition of one or more valves 12 is not limited to the specific embodiments disclosed and includes any number of housings or chambers as the circumstances require. In another embodiment, the barrier interposed between the first chamber 10 and the second chamber 20, or the second chamber 20 and the third chamber 30 can be a soluble plug or an insoluble breakable membrane.

In another embodiment, the fourth chamber 90 holds, for example, a component 80, for example, the cross linker or an enzyme. Alternatively, the fourth chamber 90 holds the diluent or the sealant matrix. Any of the chambers may hold any one or more of the components of the tissue sealant.

According to one embodiment, one or more of the first chamber 10, second chamber 20, third chamber 30 and fourth chamber 90 may hold one or more drugs, for example, antibiotics, anti-inflammatory drugs, growth factors, or other pharmaceutical or bioactive compounds.

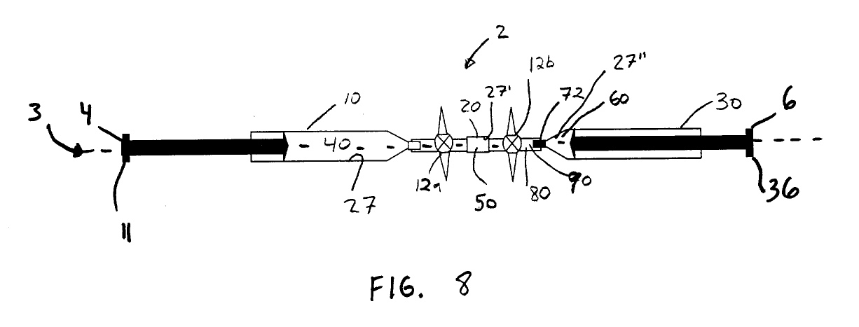

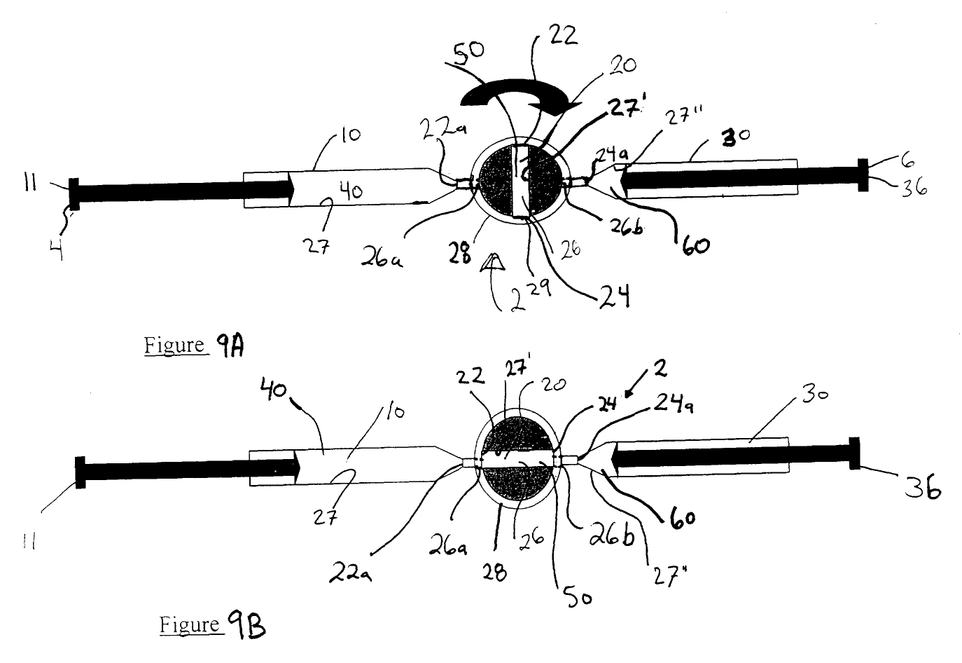

Referring now to FIG. 9A, in another embodiment, the device 2 has a valve, i.e. a stop-cock, the stop-cock comprising a rotationally mounted bore 26 and a casing member 28. The rotationally mounted bore 26, for example, a tube, is rotationally mounted in the casing member 28, the bore 26 forming the second chamber 20. The casing member 28 includes ends 22a and 24a which are in fluid communication with the first chamber 10 and the third chamber 30 respectively. The second chamber 20 is a closed chamber except for ends 22 and 24. The ends 22 and 24 are optionally in fluid communication with the first chamber 10 and the third chamber 30 respectively via bores 26a and 26b in the casing member 28. The bores 26a and 26b extend from ends 22 and 24 to ends 22a and 24a. In one embodiment, the second chamber 20 is detachably connected to the first chamber 10, and the third chamber 30, for example, by luer lock fittings that are attached to or integral with the casing member 28. The rotationally mounted bore 26 defines a lumen 27′ wherein the lumen 27′ of the rotationally mounted bore 26 can contain a component of the tissue sealant, for example, component 50. A seal 29 is located between the casing member 28 and the rotationally mounted bore 26 to prevent leakage of the component 50. In various embodiments, the seal 29 can be accomplished, for example, by a pressure fitting, or by one or more o-rings. The rotationally mounted bore 26 can rotate relative to the casing member 28 to move the second chamber 20 from the closed position shown in FIG. 9A, to the open position shown in FIG. 9B. In the open position illustrated in FIG. 9B, the lumen 27′ of the second chamber 20 is in fluid communication with the first chamber 10 and the third chamber 30 via bores 26a and 26b in the casing member 28. When the rotationally mounted bore 26 is rotated so that the second chamber 20 is in the open position, the transferor 11 and the transferor 36, for example, the plunger 11 and the plunger 36 can be used to reciprocally transfer the first component 40, the second component 50, and the third component 60 between the first chamber 10, the second chamber 20 and third chamber 30. Mixing is accomplished by reciprocally transferring the first component 40, the second component 50 and the third component 60 between the first chamber 10, the second chamber 20, and the third chamber 30 as described hereinabove. In one embodiment, one or more powders may be placed in the second chamber 20, and sealant matrix and diluent, for example, may be placed in the first chamber 10 and the third chamber 30 respectively. Alternatively, any of the sealant components can be placed in any chamber. After reciprocally transferring the contents of the housings or chambers to mix the sealant components, the tissue sealant is ready to be applied to a tissue defect.

In this aspect of the invention, the rotationally mounted bore 26 and the casing member 28 can be manufactured from, for example, one or more metals, a metal alloy, ceramic, glass, or plastics, such as polyvinylchloride, or polyethylene, preferentially, polypropylene or polycarbonate. Moreover, the diameter and length of the second chamber 20 can be optimized for peak mixing performance. In a particular embodiment, the diameter of the second chamber 20 is between 0.05 and 0.3 inches. In another embodiment, the diameter of the second chamber 20 is between 0.100 and 0.200 inches. In yet another embodiment, the diameter of the second chamber 20 is between 0.125 inches and 0.175 inches. In yet another embodiment, the length of the second chamber 20 is between 0.1 and 3 inches. In yet another embodiment, the length of the second chamber 20 is between 0.3 and 1.5 inches. In yet another embodiment, the length of the second chamber 20 is between 0.5 and 1.0 inches. In another embodiment according to the invention, the second chamber may be divided into two or more chambers by adding one or more barriers such as inert plugs or insoluble breakable membranes as described above (not shown), each chamber containing a component of the tissue sealant.

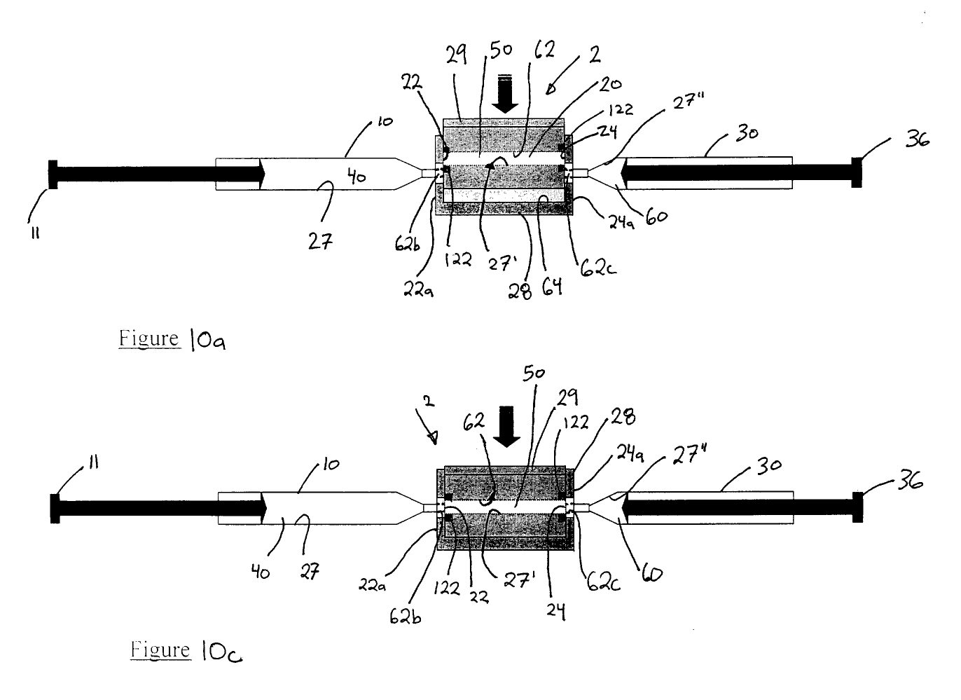

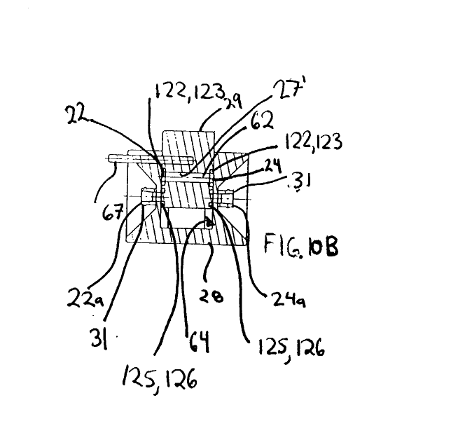

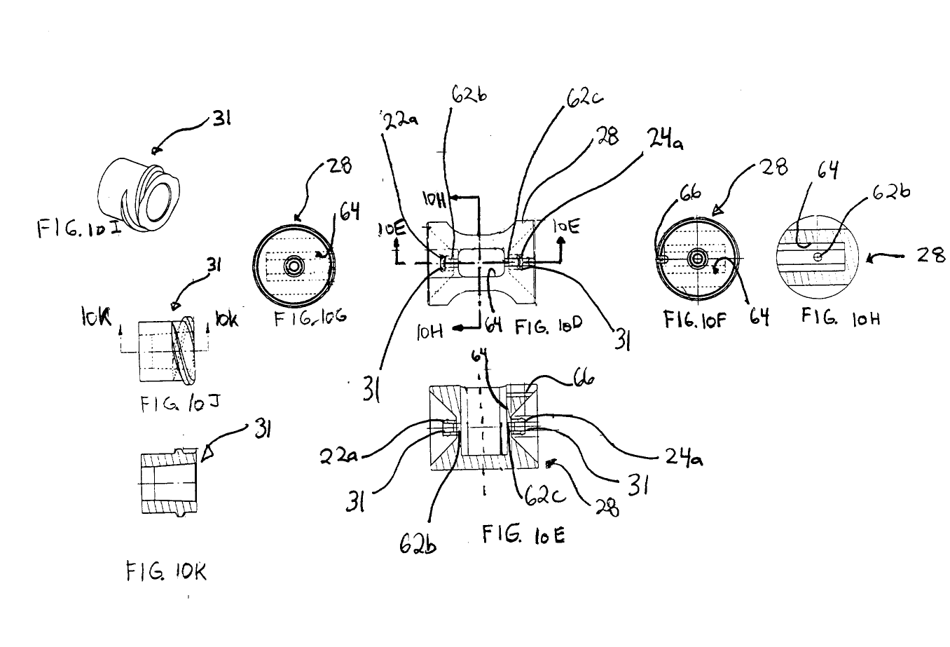

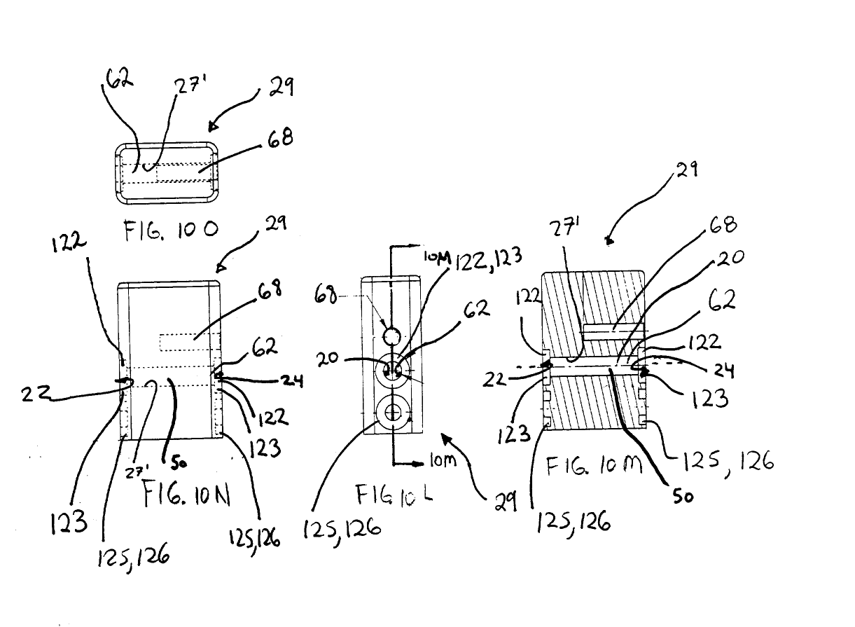

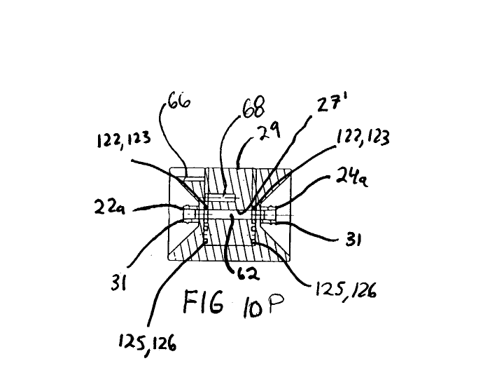

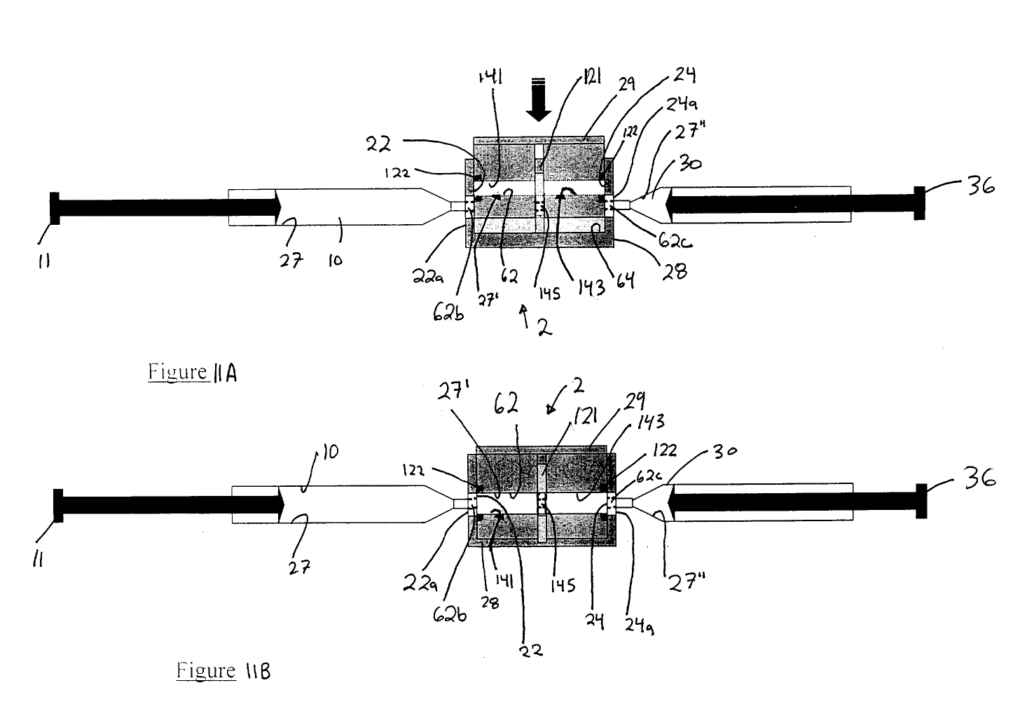

Referring now to FIG. 10A, in another embodiment, the device 2 has a slideable member 29 that can slide relative to a casing member 28 along a first axis. In operation, the slideable member 29 and the casing member 28 act together to form a valve. The slideable member 29 contains a cylindrical bore 62 disposed along a longitudinal axis that is perpendicular to the first axis. The bore 62 forms the second chamber 20. The bore 62 extends from a first end 22 of the second chamber 20, to a second end 24 of the second chamber 20. Referring to FIGS. 10B, and 10D-10H, the casing member 28 further includes a channel 64 which is longitudinally disposed along the first axis. The bore 62 can be aligned with the first chamber 10 and third chamber 30 by slideably moving the slideable member 29 in the chamber 64. The slideable member 29 and the casing member 28 can be made from any materials with appropriate mechanical strength that are also compatible with the sealant or adhesive. Specifically, materials such as, but not limited to, polycarbonate, polyethylene, nylon and polypropylene can be used.

Referring to FIGS. 10D-10H, one embodiment of the casing member 28 is shown in greater detail. The casing member 28 has a first end 22a and a second end 24a, which are in fluid communication with the first chamber 10 and the third chamber 30 respectively. Referring to FIGS. 10I-10K, the casing member 28 is connected to the first chamber 10 and the third chamber 30, for example, by luer lock fittings 31. The luer lock fittings 31 allow the casing member 28 to be connected to and detached from the first chamber 10, and the third chamber 30. Alternatively, the casing member 28 can be connected to the first chamber 10 and the third chamber 30 by a snap fit, or threading, for example. The first end 22a and the second end 24a of the casing member 28 are also in fluid communication with the channel 64 of the casing member 28 via bores 62b and 62c. In one embodiment illustrated in FIG. 10E, the top portion of the casing member 28 has a slot 66 which receives a pin 67. In use (later described), the pin 67 can prevent the slideable member 29 from sliding in the channel 64 of the casing member 28.

Referring now to FIGS. 10L-10O, the slideable member 29 is depicted in greater detail. The slideable member 29 is snuggly received in the channel 64 of the casing member 28. The bore 62 of the slideable member 29, i.e. the second chamber 20, can contain a component 50 of the tissue sealant. One or more seals 122 may be positioned between the casing member 28 and the slideable member 29 in a first circular slot 123 in the slideable member 29 to prevent leakage of the component 50. The seals can be created, for example, but without limitation, through the use of a press fit, or through the use of o-rings. A second set of seals 125 located in a second circular slot 126 in the slideable member 29 further prevents leakage of the first component 40 and the third component 60 when the slideable member 29 is in the closed position illustrated in FIG. 10B (later described). The slideable member 29 may also contain a second bore 68 which can receive the pin 67 when the second chamber 20 is in the closed position illustrated in FIG. 10B.

In use, the slideable member 29 can be moved from a closed position as shown in FIGS. 10A and 10B, to an open position shown in FIGS. 10C and 10P, by sliding the slideable member 29 along the channel 64 in the casing member 28. In the closed position as shown in FIG. 10B, the pin 67 can lock the slideable member 29, such that the slideable member 29 is prevented from sliding in the channel 64. To lock the slideable member 29, the pin 67 is inserted into the slot 66 of the casing member 28 and also into the bore 68 of the slideable member 29, which prevents movement of the slideable member 29 along the first axis. To enable movement of the slideable member 29 in the channel 64 of the casing member 28, the pin 67 is removed from the bore 68 of the slideable member 29 and the slot 66 of the casing member 28. When the slideable member 29 is actuated to slide in the channel 64 of the casing member 28, the bore 62 of the slideable member 29 may be aligned with the bores 62b and 62c and ends 22a and 24a of the casing member 28. The transferor 11 and the transferor 36, respectfully, can then be used to reciprocally transfer the first component 40, the second component 50, and the third component 60 between the housings or chambers as described hereinabove.

In one embodiment, for example, powders are placed in the second chamber 20, and a sealant matrix and diluent are placed in the first chamber 10 and the third chamber 30, respectively. Any of the tissue sealant components may be placed in any chamber. Mixing of the sealant components is accomplished by reciprocally transferring the first component 40, the second component 50, and the third component 60 between the first chamber 10, the second chamber 20, and the third chamber 30 as described hereinabove.

In this aspect of the invention, the diameter and length of the second chamber 20 can be optimized to achieve peak mixing performance. In a particular embodiment, the diameter of the second chamber 20 is between 0.05 and 0.3 inches. In another embodiment, the diameter of the second chamber 20 is between 0.100 and 0.200 inches. In another embodiment, the diameter of the second chamber 20 is between 0.125 inches, and 0.175 inches. In yet another embodiment, the length of the second chamber 20 is between 0.1 and 3 inches. In yet another embodiment, the length of the second chamber 20 is between 0.3 and 1.5 inches. In yet another embodiment, the length of the second chamber 20 is between 0.5 and 1.0 inches. In another embodiment according to the invention, the second chamber may be divided into two or more chambers by adding one or more barriers 72, for example inert plugs or insoluble breakable membranes into the second chamber 20 as described above (not shown).

In another embodiment according to the invention, illustrated in FIGS. 11A and 11B, the second housing, i.e. the second chamber 20 described above with respect to FIG. 10A can be further divided into a plurality of chambers by a stationary tab 121. For example, the stationary tab 121 can divide the second chamber 20 into a second chamber 141 and a third chamber 143. The stationary tab 121 is an elongated member that is positioned along the first axis anywhere in the slideable member 29. The stationary tab 121 includes a throughhole 145 that is aligned with ends 22a and 24a of the casing member 28. As shown in FIG. 11A, when the slideable member 29 is in the closed position, the tab 121 separates the active components, which are contained in the second chamber 141 and the third chamber 143. When the slideable member 129 is depressed into the channel 64 of the casing member 28 as shown in FIG. 11B, such that the slideable member 29 is in the open position, the chambers 10, 141, 143, and 30 align permitting the chamber 141 and the chamber 143 to communicate via the throughhole 145 in the stationary tab 121. When the chambers are aligned as shown in FIG. 11B, the components of the chambers can be reciprocally transferred between chambers 10, 141, 143, and 30 through use of the transferor 11 and the transferor 36 as described hereinabove. After reciprocally transferring the contents of the chambers, and mixing together all of the sealant components, the tissue sealant is ready to be applied to a tissue defect. The number of chambers that may be made by one or more stationary tabs is not limited to the specific embodiments disclosed and includes any number of chambers as the circumstances require.

In another embodiment, according to the invention, one or more of the chambers further holds a visualant such as a soluble dye to be used as an indicator of adequate mixing of the components to form a homogeneous mixture. In yet another embodiment, the dye in one chamber may be a different color than the dye in another chamber, for example, a blue dye in one chamber, and a yellow dye in another chamber. When the contents of the chambers are mixed together, such as the yellow and blue dye, yet a third color may appear, such as a green color, when the contents are thoroughly mixed.

In another embodiment, according to the invention, one or more of the chambers further contains an obstruction to create turbulent flow, thus enhancing the mixing efficiency of the sealant. Examples of obstructions include, but are not limited to, static mixers, restrictors, propellers, and stationary meshes.

The components of the tissue sealant may assume a variety of physical forms. For example, one component may be a powder, another a gel, and another a fluid. Alternatively, two or more components may be a fluid or a gel. The powder, gel, or fluid may be held in the first chamber 10, the second chamber 20, the third chamber 30 or any other chamber, for example, the fourth chamber 90. In a particular embodiment according to the invention, the component 40 in the first chamber 10 is a gel, the component 50 in the second chamber 20 is water, the component 60 in the third chamber 30 is a powder, and the component 80 in the fourth chamber 90 is a powder.

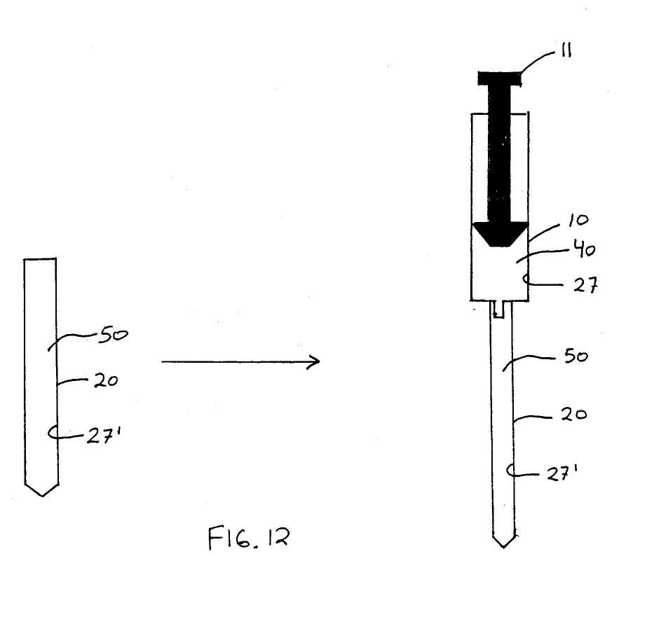

In another aspect, the invention comprises a device for mixing an adhesive, such as albumin, and EDC in a weakly active state, such as at an elevated pH. Referring now to FIG. 12, the device of this aspect of the invention includes a first chamber 10 connected to a second chamber 20. The chambers 10 and 20 can optionally be detached. The first chamber 10 holds a first component 40, such as, for example, albumin mixed with a cross-linker, for example, a carbodiimide cross-linker (EDC) to form a first mixture. The first component 40 may be alkaline at a pH at about 7-13, or more preferably at a pH at about 7-11, or most preferably at a pH at about 7-9. At this pH the albumin-EDC mixture is a semi-stable matrix that will not cure for a few minutes to a few hours. EDC reactivity is a function of pH and diminishes as pH rises. Thus, the higher the pH, the longer the time before the sealant cures. The first chamber 10 includes a transferor, such as a plunger 11. In a particular embodiment according to the invention, the first chamber 10 is a syringe comprising a barrel and a plunger.

The second chamber 20 is a tube, cylinder, or static mixer holding a second component 50. In this embodiment, the second component 50 is an acidic buffer, which lowers the pH of the first mixture. Non limiting examples of acidic buffers 50 of this invention include organic acids, inorganic acids, acid exchange resins, and ion exchange resins. More specifically, morpholinoethanesulfonic acid (MES) is an example of a buffer 50 that can be used in accordance with the invention. Other buffers 50 that can be used in accordance with the invention include citric acid, phosphate, succinates and other acidic salts. In another embodiment, the second component 50 may comprise an acid resin, such as Dowex® weakly acidic exchange resin. Optionally a first valve 12 may operatively join the first chamber 10 and the second chamber 20 (not shown).

According to this embodiment of the invention, the albumin and carbodiimide at a pH in the range of 7-13, i.e., the first component 40, are introduced into the first chamber 10. At a pH in the range of 7-13, the first mixture begins to cure at a low rate. The first chamber 10 is then joined to the second chamber 20 such that the first chamber 10 and the second chamber 20 are in fluid communication. Alternatively, the first chamber 10 and the second chamber 20 are joined before the albumin and carbodiimide mixture are added to the first chamber 10. The valve 12 optionally separates the first chamber 10 and the second chamber 20 (not shown).

The first mixture is introduced by the action of the plunger 11 from the first chamber 10 into the second chamber 20. In the second chamber 20, the first mixture is mixed with the acidic buffer 50. The buffer 50 is selected to cause the mixture resulting from the combination of the first mixture and the buffer 50 (hereinafter the second mixture) to reach a pH lower than the pH of the first mixture. For example, the pH of the first mixture may be lowered to a pH in the range of 1-7, preferably 3-7, and most preferably 5.5-6.5. The first mixture can be rapidly cured by passing it through the second chamber 20 containing the acidic buffer 50. The second mixture comprised of the first mixture mixed with the buffer 50 will cure more rapidly due to a lower pH. Accordingly, the buffer 50 that is selected is the buffer that provides the desired pH and thus the desired cure rate. As used in the context of the invention described herein, cure is defined as the amount of time necessary to produce a non-fluid, cross-linked matrix. As the first mixture passes through the acidic buffer 50 which may be coated on the inner surface of the lumen 27′ of the second chamber 20, the pH of the first mixture will be reduced thereby shortening the cure time of the sealant. Other factors will affect the cure rate, including but not limited to, the length and diameter of the second chamber 20, the concentration of the acidic buffer 50, the solubility of the buffer 50, and the stability of the sealant cross-linker matrix 40. Once the first mixture is mixed with the buffer 50, the resulting second mixture is ready to be applied to tissue as a tissue sealant.

In an alternative embodiment of this aspect of the invention, referring still to FIG. 12, the device includes a first chamber 10 and a second chamber 20. In this embodiment the first chamber 20 holds a first component 40, such as an adhesive, for example, albumin. The first chamber 10 includes a transfer means, such as a plunger 11. In a particular embodiment according to the invention, the first chamber 10 is a syringe having a barrel and a plunger. The second chamber 20 is a tube, cylinder, or static mixer and holds the second component 50. According to this embodiment of the invention, the second component is a cross-linker, for example, EDC. Optionally a first valve 12 may operatively join the first chamber 20 and the second chamber 30 (not shown).

According to this embodiment of the invention, the first chamber 10 and the second chamber 20 are joined after the albumin 40 is added to the first chamber 10. Alternatively, the first chamber 10 and the second chamber 20 are joined and after the first and second chambers are joined, albumin 40 is added to the first chamber 10.

According to one feature of this embodiment, the first component 40 is introduced from the first chamber 10 into the second chamber 20. In the second chamber 20, the first component 40 is mixed with the crosslinker 50. Once the first component 40 is mixed with the crosslinker 50, the resulting mixture is ready to be applied to tissue as a tissue sealant.

In another embodiment of the invention, the pH of the first component 40 may be acidic at about a pH of 1-7, more preferably at about a pH of 3-7 and most preferably at about a pH of 5-7. In this embodiment of the invention, the second component 50 is an alkaline buffer that adjusts the pH of the first component 40. Non limiting examples of alkaline buffers of this invention include organic bases, inorganic bases, alkaline exchange resins, or ion exchange resins.

When the first component 40 and the second component 50 are mixed as described above, the alkaline buffer 50 causes the mixture resulting from the combination of the first component 40 and the buffer 50 to reach a pH higher than the pH of the first component 40. For example, the pH may be raised to a pH in the range of 7-13, preferably 7-11, and most preferably 7-9. The first component 40 can be rapidly cured by passing it through the second chamber 20 containing the alkaline buffer. The mixture of the first component 40 and the buffer 50 will cure more rapidly due to a raised pH. Accordingly, the buffer 50 that is selected is the buffer that provides the desired pH and thus the desired cure rate. As the first component 40 passes through the alkaline buffer 50 which may be coated on the inner surface of the lumen 27′ of the second chamber, the pH of the first component 40 will be raised thereby shortening the cure time of the sealant. Other factors will affect the cure rate, including the concentration of the alkaline buffer 50, the solubility of the buffer 50, and the stability of the sealant crosslinker matrix 40. Once the first component 40 is mixed with the buffer 50, the resulting mixture is ready to be applied to tissue as a tissue sealant.

Other embodiments incorporating the concepts disclosed herein may be used without departing from the spirit and scope of the invention. The described embodiments are to be considered in all respects as only illustrative and not restrictive.

What is claimed is:

1. A device for mixing two or more components, the device comprising:
a first housing for holding a first component;
a second housing for holding a second component;
a third housing; and
a mixing element for reciprocal transfer of at least one of the first and second components between at least two of the first, second, and third housings.

2. The device of claim 1 further comprising a removable barrier interposed between at least two of the housings.

3. The device of claim 1 wherein said third housing holds a third component.

4. The device of claim 1 wherein said second housing is in fluid communication with said first housing.

5. The device of claim 1 wherein said second housing is in fluid communication with said third housing.

6. The device of claim 1 wherein said first housing is in fluid communication with said third housing.

7. The device of claim 1 wherein said mixing element comprises at least one plunger.

8. The device of claim 1 wherein said mixing element is selected from the group consisting of a piston, a bladder, a pump, a propeller, and an intraluminal ball or any combination thereof.

9. The device of claim 1 wherein at least two of said housings are detachable.

10. The device of claim 2 wherein the removable barrier comprises a valve interposed between at least two housings.

11. The device of claim 10 wherein said valve comprises a stop-cock.

12. The device of claim 1 wherein said mixing element comprises a first plunger joined to the first housing and a second plunger joined to the third housing for reciprocally transferring at least one of the first and second components between the first housing and the third housing through the second housing.

13. The device of claim 1 further comprising a slideably mounted member, wherein the slideably mounted member contains a bore, the bore forming the second housing.

14. The device of claim 13 wherein the second housing is further divided into a plurality of housings.

15. The device of claim 1 wherein a soluble plug divides one or more of the first, second, and third housings.

16. The device of claim 1 wherein an insoluble breakable membrane divides one or more of the first, second, and third housings.

17. The device of claim 1 wherein one or more housings comprise an obstruction to create turbulent flow to enhance mixing efficiency.

18. The device of claim 1 wherein at least two of the housings comprise a syringe.

19. The device of claim 2 wherein the removable barrier comprises a soluble plug.

20. The device of claim 2 wherein the removable barrier comprises an insoluble breakable membrane.

21. A device for preparing a tissue sealant, comprising:
a first chamber comprising a first lumen for holding a first component;
a second chamber comprising a second lumen for holding a second component, said second lumen in fluid communication with said first lumen of said first chamber;
a third chamber comprising a third lumen, said third lumen in fluid communication with said second lumen of said second chamber; and
a transferor for reciprocal transfer of at least one of the first and second components between at least two of the first, second, and third chambers.

22. A system for preparing a tissue sealant with a predetermined cure rate, comprising:
a first chamber comprising a lumen;
a first mixture, held in the first chamber, comprising an adhesive matrix and a cross-linker;
a second chamber detachably fitted to the first chamber;
a second component held in the second chamber for adjusting the pH of the first mixture.

23. The system of claim 22 wherein the first mixture held in the first chamber is mixed with the second component held in the second chamber to form a second mixture, and wherein the second component adjusts the pH of the second mixture to form a sealant comprising the predetermined cure rate.

24. The system of claim 22, wherein the second component held in the second chamber comprises a buffer.

25. The buffer of claim 24, wherein the buffer is selected from the group consisting of an ion exchanging resin, organic acids, inorganic acids, acid exchange resins, organic bases, inorganic bases and alkaline exchange resins.

26. The buffer of claim 24 wherein the buffer is selected from citric acid, phosphate, succinate, acidic salts, and morpholinoethanesulfonic acid.

27. The system of claim 22 wherein the pH of the first mixture is in the range of about 7-13.

28. The system of claim 22 wherein the first mixture comprises a carbodiimide crosslinker.

29. The system of claim 23 wherein the second component lowers the pH of the second mixture to control the cure rate of the second mixture.

30. The system of claim 22 wherein the pH of the first mixture is in the range of about 1-7.

31. The system of claim 23 wherein the second component raises the pH of the second mixture to control the cure rate of the second mixture.

32. The system of claim 22 wherein the lumen of the second chamber is coated with the second component.

33. A method for preparing a tissue sealant with a predetermined cure rate, comprising:
fitting a first chamber holding a first mixture comprising an adhesive matrix and a cross-linker at about pH 7-13 to a second chamber,
introducing the first mixture from the first chamber into the second chamber holding a buffer, wherein the buffer adjusts the pH of the first mixture to a predetermined level; and
mixing the first mixture and the buffer in the second chamber to form a sealant having a predetermined cure rate.
